(19) United States
(12) Patent Application Publication
(10) Pub. No.: US 2020/0411174 A1
(43) Pub. Date: Dec. 31, 2020

(54) DOCUMENT MANAGEMENT SYSTEM

(71) Applicant: IRYOU JYOUHOU GIJYUTU KENKYUSHO CORPORATION, Fukuoka (JP)

(72) Inventor: Shinkichi HIMENO, Fukuoka (JP)

(21) Appl. No.: 17/017,148

(22) Filed: Sep. 10, 2020

Related U.S. Application Data
(63) Continuation-in-part of application No. 16/098,967, filed on Nov. 5, 2018.

(30) Foreign Application Priority Data
May 12, 2016 (JP) 2016-095758

(51) Int. Cl. G16H 40/20; G06F 16/93; G16H 10/60; G06Q 10/10

(52) U.S. Cl. CPC G16H 40/20; G06Q 10/105; G16H 10/60; G06F 16/93

(57) ABSTRACT

[Problem] To promptly notify a change in an instruction document or record document pertaining to medical service, etc., to a staff who needs to know the change and eliminate the need for always monitoring changes, thereby making it possible to eliminate oversight and effectively utilize time accordingly. [Solution] A document management system provided with a case management means for managing a plurality of cases, a document category management means for managing a plurality of document categories, and a per-document category document recording means for recording a document for each document category. The document management system is provided with a trigger condition setting means for setting a trigger condition for triggering notification of the processing state of a document, a trigger condition applicable document detection means for detecting a document that falls under the trigger condition, a notified party setting means for setting a notified party to which the document detected to fall under the trigger condition is notified, and a trigger applicable document notification means for notifying the trigger applicable document detected to fall under the trigger condition by the trigger condition applicable document detection means to the notified party set by the notified party setting means.

### TECHNICAL FIELD

The present invention relates to a document management system that efficiently manages documents for instructions and records necessary when a large number of staff members work in parallel on a plurality of cases via a computer network, and especially to a document management system for an electronic medical record or the like which manages instructions and record documents relating to treatment or nursing of a patient at hospitals and the like.

### BACKGROUND ART

A large number of types of jobs such as medical doctors, nurses, caregivers, rehabilitators, pharmacists, and laboratory technicians are involved in the treatment and the like of patients. On the basis of a doctor's instruction document, a person of a type of job in charge confirms the instruction to prepare for and execute and creates a record document. Taking a blood examination involving multiple types of jobs as an example, a nurse draws blood and passes a sample to a laboratory technician and in turn the laboratory technician executes the examination and records a result thereof in a report document.

When there are multiple wards and examination instruction documents are sent from each ward, the laboratory technician creates his/her own work schedule (worksheet) and notifies each ward thereof. A nurse in a ward creates his/her own schedule based on schedules sent from each of other departments such as radiation department or physiological examination department so as to draw blood on the basis of this schedule and transfer a sample to the examination room.

In this manner, it is necessary for a large number of types of jobs to process diverse instruction documents in parallel in a distributed manner and keep results thereof in record documents. In working, it is essential to create a worksheet for each patient and for each type of job and proceed neatly with work. In addition, in a case where, for example, a work crosses multiple types of jobs, it is necessary to grasp the current progress status of the work such that the omission and duplication of implementation of the work are prevented.

Prior art literatures related to this application are as follows.

### CITATION LIST

### Patent Literature

Patent Literature 1: JP 11-066213 A

Patent Literature 2: JP 2012-033095 A

In Patent Literatures 1 and 2, related documents such as confirmation, implementation, and result reports are created for a medical instruction document such that it can be confirmed up to which stage the instruction has been implemented by which staff member. In addition, a worksheet is automatically created by consolidating the instruction documents for each ward and for each service department to make it possible to view the progress status of each instruction as a list by a technique such as changing the color of the document according to the creation stage of the related document.

### SUMMARY OF INVENTION

### Technical Problem

The patient's condition always changes. In accordance therewith, the instruction of medical care always changes.

That is, a new instruction document is suddenly created, or conversely, the content of the instruction document that has been issued is changed or deleted.

If these changes are missed, medical errors such as omission of implementation are brought about. Accordingly, medical staff members of each type of job need to constantly monitor whether no addition, change, or deletion has been made on instruction content, on a screen of the worksheet of electronic medical records. For this reason, a great deal of time has been wasted.

For blood examination or the like, it takes time to implement the examination and obtain the examination result since the instruction is issued.

In addition, for the instruction of the periodical examination, the result report document is created before a doctor who issued the instruction notices that. For this reason, many of the examinations that have been done carefully are not referred to in a timely manner and, in extreme cases, end up without being seen by the doctor at all. According to the result of a survey actually conducted at a US hospital, 40% of blood examinations, X-ray examinations, and the like end up without being referred to at the appropriate time.

Furthermore, it is estimated that this oversight will account also for 40% as a cause of medical accidents. Moreover, in order to avoid medical accidents due to such oversight, when an abnormal value is observed in a result of blood drawing or the like, the medical staff member is forced to confirm whether the doctor in charge has already referred to this result, which produces extra work.

The present invention has been made in order to solve such conventional difficulties and it is an object of the present invention to remove oversight by promptly notifying a staff member who needs to know a change in, for example, a medical instruction document or record document of this change to eliminate the need for constant monitoring of the change, making it possible to make effective use of the time saved therefrom, and furthermore, to provide a document management system in which a report on a result or the like of examination implemented carefully is surely referred to by a medical doctor and the like in a timely manner and the fact that the report has been referenced is promptly notified to a related medical staff member.

### Solution to Problem

As a means for achieving the above object, a document management system according to claim 1 is a document management system including: a server apparatus that includes a processor and communicates with a database, wherein the processor executes:

associating each of a plurality of cases with a document;

associating each of a plurality of document categories with a document;

recording a document on the database for each document category,

the processor further executes:

setting a trigger condition serving as a trigger on the database for notifying a processing state of a document;

detecting a document on the database corresponding to the trigger condition;

registering a terminal of a notification destination to be notified of a document detected as corresponding to the trigger condition in accordance with the document category corresponding to a document;

notifying the terminal of the notification destination of a trigger corresponding document detected as corresponding to the trigger condition;

registering as a range on the database for applying the trigger condition, one of the following: (1) all of documents belonging to any of the document categories corresponding to a medical activity or an inspection activity and related documents; (2) a particular document group corresponding to a type of a patient among documents belonging to one of the document categories and a related document; and (3) a particular document corresponding to a particular patient among documents belonging to one of the document categories and a related document; and registering, as the trigger condition, on the database, at least one processing state among processing states of creation; update; and deletion of a document belonging to a range set by the document range setting unit; and creation; update; and deletion of a related document of the document.

A document management system according to claim 2 is the document management system according to claim 1, in which the processor executes registering, as a notification destination, at least the terminal of a particular staff member, the terminal of a staff member belonging to a particular type of job, the terminal of a staff member belonging to a particular work place, and any combination of the terminals of the staff members.

A document management system according to claim 3 is the document management system according to claim 1, in which the processor further executes cooperating with any one of a mail system or a sticky note system within the present document management system, an in-hospital mail system outside the present document management system, and a public mail system outside a hospital, or any combination of the foregoing systems.

A document management system according to claim 4 is the document management system according to claim 1, in which the processor further executes:

referring the trigger corresponding document in accordance with a request from the terminal; and

sending the referred trigger corresponding document to the terminal as the notification destination.

A document management system according to claim 5 is the document management system according to claim 1, in which the processor further executes receiving a response from the terminal as the notification destination, wherein the response that indicates a complete of display processing of the trigger corresponding document send from the server apparatus on the terminal as the notification destination.

A document management system according to claim 6 is the document management system according to claim 5, in which the processor further executes notifying the terminal of the notification destination of a trigger corresponding document in case of that the server apparatus does not receive the response from the terminal of the notification destination during a predetermined time from when the trigger corresponding document is detected.

### Advantageous Effects of Invention

The present invention remove oversight by promptly notifying a staff member who needs to know a change in, for example, a medical instruction document or record document of this change to eliminate the need for constant monitoring of the change, making it possible to make effective use of the time saved therefrom, and furthermore, can provide a document management system in which a report on a result or the like of examination implemented carefully is surely referred to by a medical doctor and the like in a timely manner and the fact that the report has been referenced is promptly notified to a related medical staff member.

### BRIEF DESCRIPTION OF DRAWINGS

FIG. 1 is an explanatory diagram of a patient ID master as a case managing unit.

FIG. 2 is an explanatory diagram of a document category managing unit.

FIG. 3 is an explanatory diagram of a document-category-specific recording unit.

FIG. 4 is an explanatory diagram of related document creation caused in the course of progression of an instruction document process and a notification destination that is triggered by the related document creation.

FIG. 5 is an explanatory diagram of a trigger condition and notification destination setting table.

FIG. 6 is an example of a notification document by a trigger corresponding document notifying unit.

FIG. 7 is a hardware configuration diagram of the document management system

### DESCRIPTION OF EMBODIMENTS

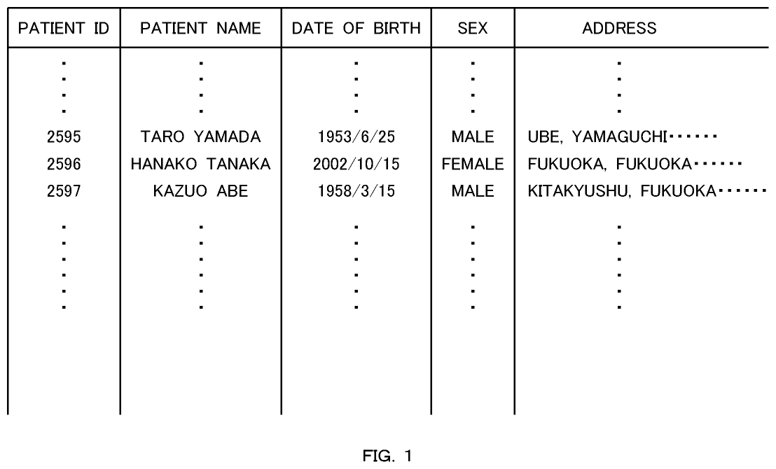

In FIG. 1, a patient ID master is taken here as an example of a case managing unit.

A unique management number (ID number) is assigned to each case or patient and all instruction documents and record documents are managed in association with this number. Personal information such as patient name and date of birth is also managed in this patient ID master.

Although medical records will be described below as an example, it goes without saying that the present invention is not restricted to electronic medical records but can also be applied to document management for project promotion, and the like.

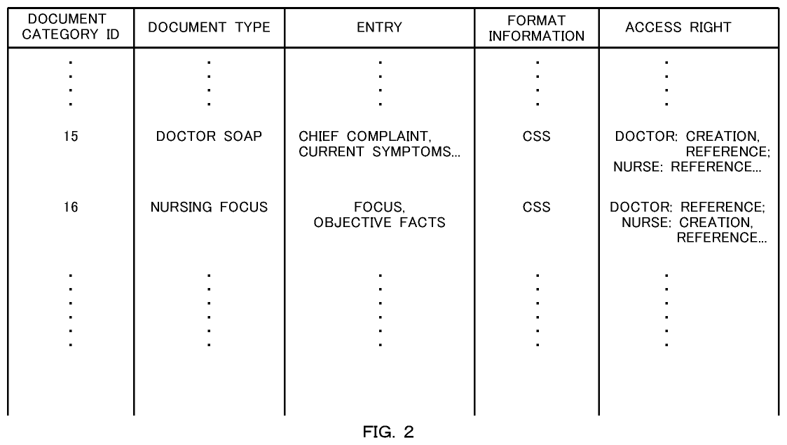

FIG. 2 is an explanatory diagram of a document category managing unit.

IDs of document categories created for each case (patient's medical record in this example), document type names, entries and format information included in documents, access rights of staff members, and the like are centrally managed for each document category.

Entries, formats, access rights, and the like are recorded as objects of an extensible markup language (XML) format in this example, but the present invention is not restricted thereto. A JavaScript object notation (JSON) format or the like may be employed as the expression format and the recording form is also optional, as long as the object of the present invention is met. Records may be composed for each entry to be recorded and managed using a relational database.

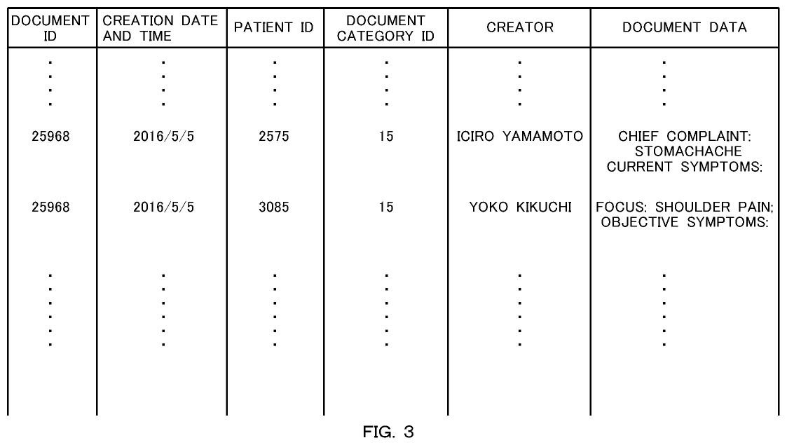

FIG. 3 is an explanatory diagram of a document-category-specific recording unit.

In this example, by allocating the ID of the document category, records are centrally managed for each document category entirely.

Search data such as a document ID as a serial number for management, a document creation date and time, a patient ID, a document category ID, a creator, and the like and object data in the XML format, which is the entity of document data, are recorded for each document.

As in the case of FIG. 2, in recording the document data in this case, the JSON format or the like may be employed as the expression format and the recording form is also optional. Records may be composed for each entry to be recorded and managed using a relational database.

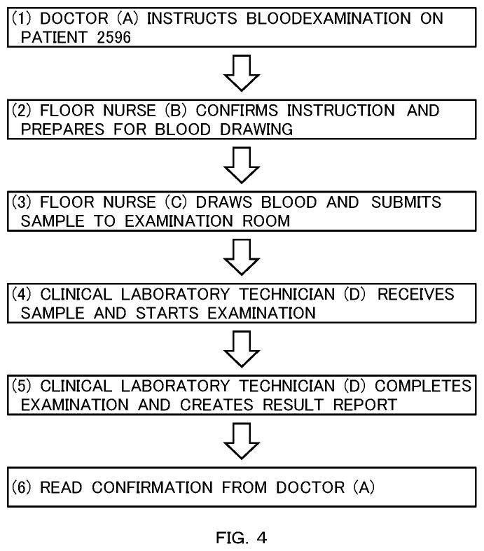

In FIG. 4, a flow of process and setting of a trigger condition and a notification destination will be described taking blood examination as an example.

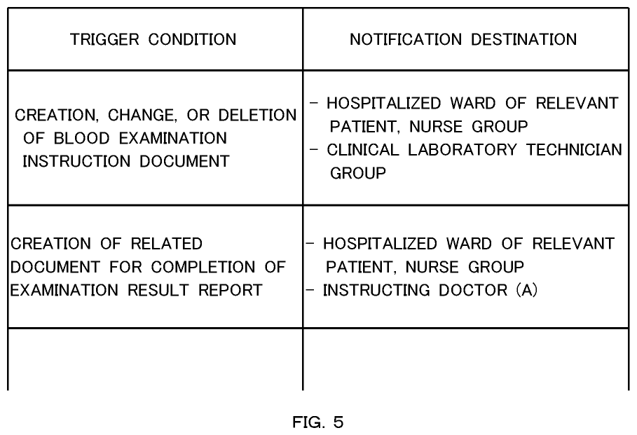

Here, FIG. 5 is a trigger condition and notification destination setting table created by a trigger condition setting unit and a notification destination setting unit.

When a document or a related document set by the trigger condition setting unit is created, changed, or deleted (document processing state designating unit), a trigger is activated and a notification is made.

(1) A doctor (A) creates a blood drawing instruction document for a patient ID 2596. The creation of this document is notified to a nurse group of a relevant ward where the patient is hospitalized, which is in charge of blood drawing, and a clinical laboratory technician group in charge of the examination (FIG. 5, the first row).

With this procedure, the floor nurses and the clinical laboratory technicians no longer need to constantly confirm whether an instruction document has been newly created or whether no change or deletion has been made, on a worksheet screen or the like, and can quickly begin taking actions such as preparation. At the same time, the risk of omission of implementation resulted from oversight is drastically reduced.

(2) Among the floor nurse group that was given the notification, a nurse (B) confirms the instruction and starts preparation for blood drawing, while creating a related document for instruction confirmation.

(3) A nurse (C) in charge of the relevant patient draws blood to deliver a sample obtained by blood drawing to a clinical examination room and at the same time creates a related document for blood drawing completion.

In this case, a triggered notification is not necessary because the delivery of the sample itself also serves as a notification.

(4) A clinical laboratory technician (D) who received the sample begins examining the sample.

(5) When the examination of the sample is finished, the clinical laboratory technician (D) inputs a result report document to an electronic medical record and also creates a related document for completion of the clinical examination.

The creation of this related document is notified to the doctor (A) who issued the instruction and the nurse group of the relevant ward according to the second row of FIG. 5. With this procedure, even if there is a time lag between the instruction creation and the result report, the result is referred to immediately without oversight and it is possible to contribute to the creation of an effective treatment policy.

(6) The doctor (A) who was given the notification opens the medical record of the relevant patient to refer to the examination result and also creates a related document for the fact that the examination result has been referred to.

This decreases the workload of floor nurses who had previously taken time to confirm whether the doctor had seen the results.

The trigger condition and notification destination setting table may be set only for a particular document. Usually, however, if the trigger condition and notification destination setting table is set by standard at the stage when the instruction document is created, for all of documents belonging to a certain document category and the related documents (for example, a blood examination instruction), or a particular document group and the related document (for example, documents relating to patients being hospitalized), or the like, this is useful as setting omissions are removed.

The notification destination is set by the notification destination setting unit. The notification destination can be set for a particular staff (the doctor (A) in this example), a staff member belonging to a particular type of job (nurse in this example), a staff member belonging to a particular work place (the ward where the patient is hospitalized in this example), and any combination of the above staff members.

Also in setting the notification destination as in the case of the trigger condition, if the notification destination is set by standard at the stage when the instruction document is created, for all of documents belonging to a certain document category and the related documents (for example, a blood examination instruction), or a particular document group and the related document (for example, documents relating to patients being hospitalized), or the like, this is useful as setting omissions are removed.

As described above, if the trigger condition and notification destination setting table is created every time an instruction document is newly created, a range for checking whether the trigger condition is satisfied when the state changes, such as when a certain related document is created, (trigger condition corresponding document detecting unit) is always maintained within a relevant document and the related document thereof, and the trigger condition and notification destination setting table set for the relevant document, such that the trigger condition can be checked in a minimum search range.

Nowadays when the performance of a computing device is improved, even a case where the trigger condition and notification destination setting table is separated to be managed and all documents are searched is also included in the scope of the present invention.

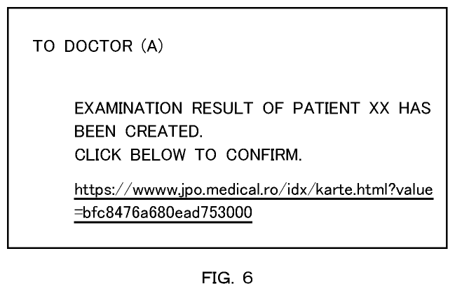

FIG. 6 is an example of the notification document transferred to a notification destination when it is determined that the trigger condition is satisfied. A script is embedded in the uniform resource locator (URL) and, when clicked, the electronic medical record of the relevant patient opens and the result of the blood examination can be referred to (referring unit for the trigger corresponding document).

In addition to simply displaying the electronic medical record of the relevant patient on a monitor screen in a usual manner, in this example, it is also useful to display the blood examination result or to create the related document for read confirmation in FIG. 4, by devising that script of the URL.

For the notification document, any of a message system or a sticky note system within an electronic medical record system, an e-mail system not included in the main body of the electronic medical record but running within a hospital, or an external e-mail system provided to the public may be used. However, the external e-mail system has security difficulties such as viruses and eavesdropping and it is thus preferable to use messaging/sticky note/e-mail system closed in the hospital.

Although an embodiment has been described so far, specific structures of the present invention are not limited to the above embodiment, and design changes and the like within the scope of the invention are included in the present invention.

For example, in the description of FIG. 4 and the following drawings, the present invention has been described based on the related document created according to the progression of the work and considered to be most useful. However, instead of creating the related document, a checklist or the like may be provided in the instruction document such that a check is put therein according to the working stage and this check put therein is used as a trigger. In addition, in FIG. 4, although it is assumed that the related document for read confirmation is created, a read confirmation function equipped in a general mail system may be used in the result report creation notification mail at the previous stage.

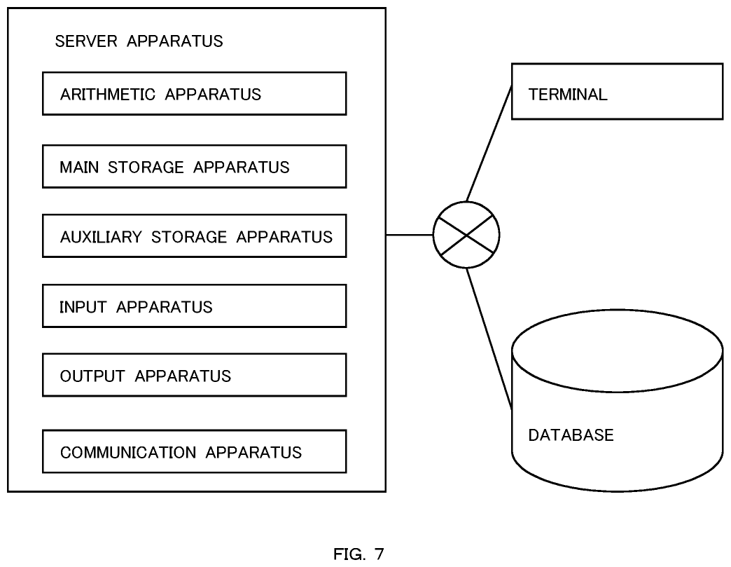

As in the case of FIG. 7, in an embodiment, the document management system includes the server apparatus, the database and the terminal. The server apparatus, the database and the terminal connected through network. The server apparatus is a prior computer. The server apparatus includes: an arithmetic apparatus including the processor, a main storage apparatus, an auxiliary storage apparatus, input apparatus, output apparatus, and communication apparatus. The arithmetic apparatus, the main storage apparatus, the auxiliary storage apparatus, input apparatus, output apparatus, the communication apparatus connected through a bus interface. The arithmetic apparatus includes the processor that can execute an instruction set. The main storage apparatus includes a volatile memory such as a random access memory (RAM). The auxiliary storage apparatus includes a recording medium such as a nonvolatile memory, and a recording method thereof is not limited. The recording medium indicates a hard disk drive (HDD) or a solid state drive (SSD), for example. The input apparatus is, for example, a keyboard device. The output apparatus includes, for example, display as a liquid crystal panel. The communication apparatus is a network interface can connected to network. The processor of the server apparatus executes the function of the units of the document management system including: case managing unit, document category managing unit, document category specific document recording unit, trigger condition setting unit, trigger condition corresponding document detecting unit, notification destination setting unit, and trigger corresponding document notifying unit or the like. The database is composed of the auxiliary storage apparatus of the server apparatus or the an auxiliary storage apparatus independent from the server apparatus. The database stores information managed by the document management system. The terminal is a prior computer including a processor.

FIG. 1

### PATIENT ID

### PATIENT NAME

### DATE OF BIRTH

### SEX

### ADDRESS

### TARO YAMADA

### MALE

### UBE, YAMAGUCHI

### HANAKO TANAKA

### FEMALE

### FUKUOKA, FUKUOKA

### KAZUO ABE

### KITAKYUSHU, FUKUOKA

FIG. 2

### DOCUMENT CATEGORY ID

### DOCUMENT TYPE

### ENTRY

### FORMAT INFORMATION

### ACCESS RIGHT

### DOCTOR SOAP

### CHIEF COMPLAINT, CURRENT SYMPTOMS

### DOCTOR: CREATION, REFERENCE; NURSE: REFERENCE

### NURSING FOCUS

### FOCUS, OBJECTIVE FACTS

### DOCTOR: REFERENCE; NURSE: CREATION, REFERENCE

FIG. 3

### DOCUMENT ID

### CREATION DATE AND TIME

### PATIENT ID

### DOCUMENT CATEGORY ID

### CREATOR

### DOCUMENT DATA

### ICHIRO YAMAMOTO

### CHIEF COMPLAINT: STOMACHACHE; CURRENT SYMPTOMS:

### YOKO KIKUCHI

### FOCUS: SHOULDER PAIN; OBJECTIVE SYMPTOMS:

FIG. 4

### (1) DOCTOR (A) INSTRUCTS BLOOD EXAMINATION ON PATIENT 2596

### (2) FLOOR NURSE (B) CONFIRMS INSTRUCTION AND PREPARES FOR BLOOD DRAWING

### (3) FLOOR NURSE (C) DRAWS BLOOD AND SUBMITS SAMPLE TO EXAMINATION ROOM

### (4) CLINICAL LABORATORY TECHNICIAN (D) RECEIVES SAMPLE AND STARTS EXAMINATION

### (5) CLINICAL LABORATORY TECHNICIAN (D) COMPLETES EXAMINATION AND CREATES RESULT REPORT

### (6) READ CONFIRMATION FROM DOCTOR (A)

FIG. 5

### TRIGGER CONDITION

### NOTIFICATION DESTINATION

### CREATION, CHANGE, OR DELETION OF BLOOD EXAMINATION INSTRUCTION DOCUMENT

HOSPITALIZED WARD OF RELEVANT PATIENT, NURSE GROUP
CLINICAL LABORATORY TECHNICIAN GROUP

### CREATION OF RELATED DOCUMENT FOR COMPLETION OF EXAMINATION RESULT REPORT

INSTRUCTING DOCTOR (A)

FIG. 6

### TO DOCTOR (A)

### EXAMINATION RESULT OF PATIENT XX HAS BEEN CREATED. CLICK BELOW TO CONFIRM.

1. A document management system including: a server apparatus that includes a processor and communicates with a database, wherein the processor executes:
associating each of a plurality of cases with a document;
associating each of a plurality of document categories with a document;
recording a document on the database for each document category,
the processor further executes:
setting a trigger condition serving as a trigger on the database for notifying a processing state of a document;
detecting a document on the database corresponding to the trigger condition;
registering a terminal of a notification destination to be notified of a document detected as corresponding to the trigger condition in accordance with the document category corresponding to a document;
notifying the terminal of the notification destination of a trigger corresponding document detected as corresponding to the trigger condition;
registering as a range on the database for applying the trigger condition, one of the following: (1) all of documents belonging to any of the document categories corresponding to a medical activity or an inspection activity and related documents; (2) a particular document group corresponding to a type of a patient among documents belonging to one of the document categories and a related document; and (3) a particular document corresponding to a particular patient among documents belonging to one of the document categories and a related document; and
registering, as the trigger condition, on the database, at least one processing state among processing states of creation; update; and deletion of a document belonging to a range set by the document range setting unit; and creation; update; and deletion of a related document of the document.

2. The document management system according to claim 1, in which the processor executes registering, as a notification destination, at least the terminal of a particular staff member, the terminal of a staff member belonging to a particular type of job, the terminal of a staff member belonging to a particular work place, and any combination of the terminals of the staff members.

3. The document management system according to claim 1, in which the processor further executes cooperating with any one of a mail system or a sticky note system within the present document management system, an in-hospital mail system outside the present document management system, and a public mail system outside a hospital, or any combination of the foregoing systems.

4. The document management system according to claim 1, in which the processor further executes:
referring the trigger corresponding document in accordance with a request from the terminal; and
sending the referred trigger corresponding document to the terminal as the notification destination.

5. The document management system according to claim 1, in which the processor further executes
receiving a response from the terminal as the notification destination, wherein the response that indicates a complete of display processing of the trigger corresponding document send from the server apparatus on the terminal as the notification destination.

6. The document management system according to claim 5, in which the processor further executes
notifying the terminal of the notification destination of a trigger corresponding document in case of that the server apparatus does not receive the response from the terminal of the notification destination during a predetermined time from when the trigger corresponding document is detected.
